(19) United States
(12) Patent Application Publication
(10) Pub. No.: US 2012/0165602 A1
(43) Pub. Date: Jun. 28, 2012

(54) Pelvic Tissue Plication System

(75) Inventor: Brian J. Nissen, Jordan, MN (US)

(73) Assignee: AMS Research Corporation, Minnetonka, MN (US)

(21) Appl. No.: 13/335,557

(22) Filed: Dec. 22, 2011

Related U.S. Application Data
(60) Provisional application No. 61/426,175, filed on Dec. 22, 2010.

(51) Int. Cl. A61F 2/02

(52) U.S. Cl. 600/30

(57) ABSTRACT

A bladder plication system to treat ISD in a patient is provided. The device can include a needle introducer device, and a plication device. The needle introducer device is advanced through the bladder, with the plication device contained and deployable within the needle introducer device. Once inserted in the bladder and positioned, the needle is passed through portions of the bladder wall. The plication device is then deployed through a distal end of the introducer device, wherein the bladder wall is folded near the inner sphincter to reduce forces on the bladder and resolve the patient's SUI.

### PRIORITY

This application claims priority to and the benefit of U.S. Provisional Patent Application No. 61/426,175, filed Dec. 22, 2010 and entitled “Pelvic Tissue Plication System,” which is incorporated herein by reference in its entirety.

### FIELD OF THE INVENTION

The present invention relates generally to surgical methods and apparatus and, more specifically, to a bladder plication device system adapted to treat intrinsic sphincteric deficiency.

### BACKGROUND OF THE INVENTION

Pelvic health for men and women is a medical area of increasing importance, at least in part due to an aging population. Examples of common pelvic ailments include incontinence (e.g., fecal and urinary), pelvic tissue prolapse (e.g., female vaginal prolapse), and conditions of the pelvic floor.

Urinary incontinence can further be classified as including different types, such as stress urinary incontinence (SUI), urge urinary incontinence, mixed urinary incontinence, among others. Other pelvic floor disorders include cystocele, rectocele, enterocele, and prolapse such as anal, uterine and vaginal vault prolapse. A cystocele is a hernia of the bladder, usually into the vagina and introitus. Pelvic disorders such as these can result from weakness or damage to normal pelvic support systems.

Urinary incontinence can be characterized by the loss or diminution in the ability to maintain the urethral sphincter closed as the bladder fills with urine. Male or female stress urinary incontinence (SUI) generally occurs when the patient is physically stressed.

In its severest forms, vaginal vault prolapse can result in the distension of the vaginal apex outside of the vagina. An enterocele is a vaginal hernia in which the peritoneal sac containing a portion of the small bowel extends into the rectovaginal space. Vaginal vault prolapse and enterocele represent challenging forms of pelvic disorders for surgeons. These procedures often involve lengthy surgical procedure times.

Urinary incontinence can be characterized by the loss or diminution in the ability to maintain the urethral sphincter closed as the bladder fills with urine. Male or female stress urinary incontinence (SUI) occurs when the patient is physically stressed.

Intrinsic Sphincteric Deficiency (ISD) is a condition resulting from an intrinsic defect in the sphincteric mechanism that results in an open bladder neck. This, along with urethral mobility, can be caused by damage to the pelvic floor and urethral ligaments, which can result in SUI.

There is a need for a device and surgical procedure designed to treat such pelvic orders, including ISD in a patient.

### SUMMARY OF THE INVENTION

The present invention describes various embodiments of a bladder plication system to treat ISD in a patient. The system can include a needle introducer device, and a plication device. The needle introducer device is advanced into the bladder, via the urethra, with the plication device deployable from within the needle introducer device. Once inserted in the bladder and positioned, the needle is passed through a portion of the bladder wall at a first location and then back again into the bladder through a second location of the bladder wall proximate the first location. The distance between the first location and the second location generally defined the size of the plication, e.g., the size of the bladder wall fold brought together. The plication device is then deployed through a distal end of the introducer device, wherein the bladder wall is folded near the inner sphincter to reduce forces on the bladder and resolve the patient's SUI.

The plication device can be constructed in a generally rivet-shaped configuration to define a first head portion, an intermediate body member, and a second head portion. The head portions and/or the intermediate body member can be constructed of a polymer metal material such the head portions are hingedly or pivotally displaceable or movable relative to the body member. Such a hingable configuration facilitates substantial collapse of the plication device onto itself to facilitate deployment and placement.

In certain embodiments, the plication device can include one or more adjustment features, enabling a user (e.g., physician) to adjust the spacing between the respective head portions to correspondingly adjust the compression on the tissue trapped therebetween during the plication folding process, and to allow for various sized tissue or bladder wall folds during the procedure.

### BRIEF DESCRIPTION OF THE DRAWINGS

FIG. 1 illustrates the bladder and associated anatomy.

FIGS. 2-6 show plication devices and corresponding features or structures in accordance with embodiments of the present invention.

FIG. 7 shows a needle introducer and deployment device in accordance with embodiments of the present invention.

FIGS. 8-11 show a plication or folding procedure of the bladder wall using a plication device in accordance with embodiments of the present invention.

### DETAILED DESCRIPTION OF PREFERRED EMBODIMENTS

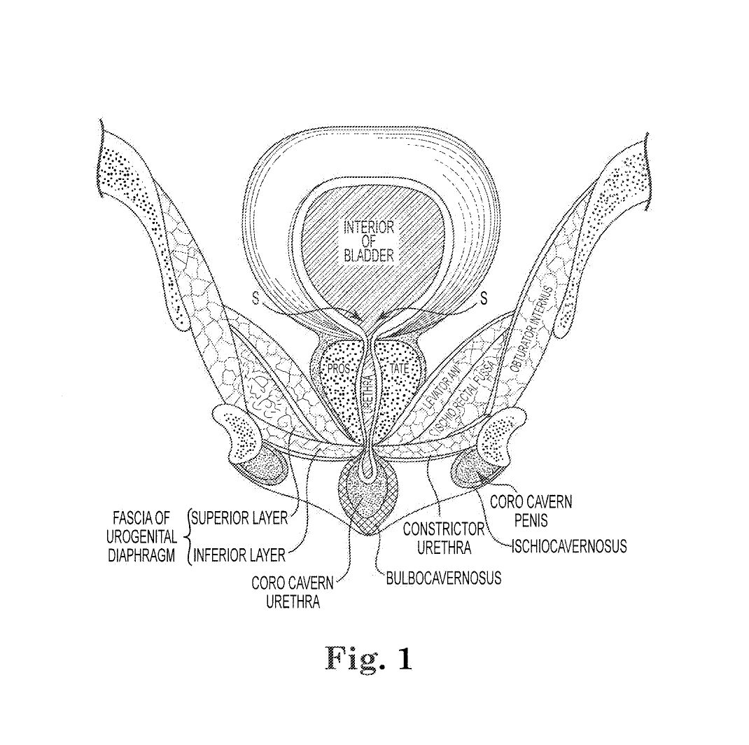

Referring generally to FIG. 1, a diagram is shown of the human bladder and associated anatomy, including the inner or internal urethral sphincter S. The internal sphincter muscle is located at the junction of the urethra with the urinary bladder and when functioning properly is the primary muscle for prohibiting the release of urine. The sphincter is made up of a ring-like band of muscle fibers that are under involuntary or autonomic control, with the sphincter normally in a closed position and needing stimulation to open. An intrinsic defect in the sphincteric mechanism can result in an open bladder neck and, thus, SUI.

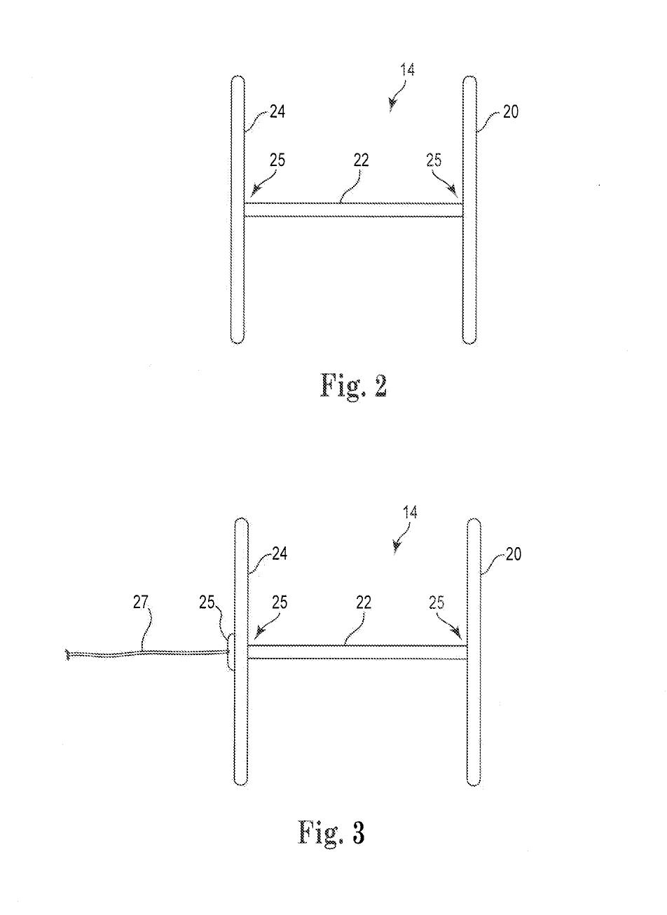

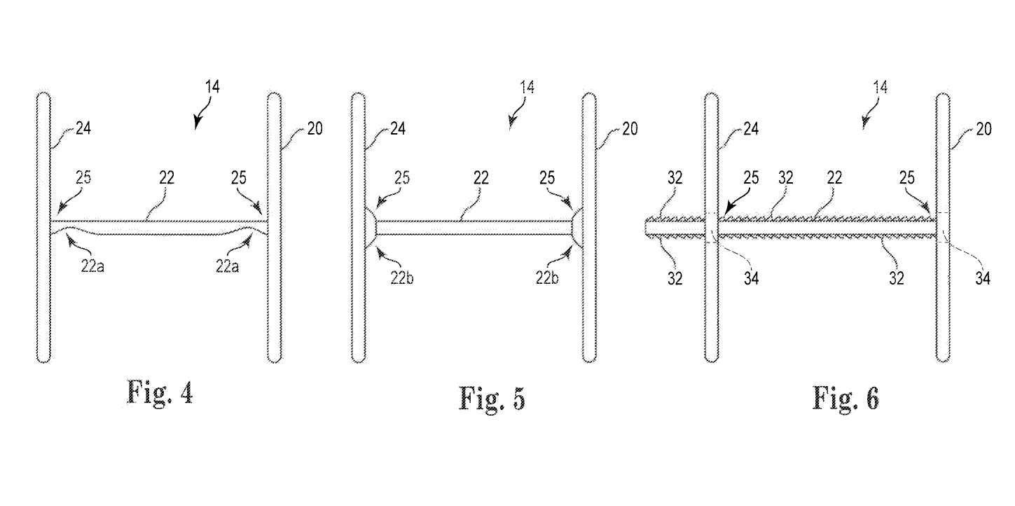

Referring generally FIGS. 2-11, various embodiments of a bladder plication system 10 are disclosed. In general, the plication system 10 can include at least one needle introducer device 12 and at least one plication device 14. Various portions of the systems 10 can be constructed of metal or polymer materials, such as polypropylene, polyethylene, fluoropolymers, Nitinol or like compatible materials. The plication device 14 is generally deployed into the bladder via the urethra to plicate or fold one or more portions of the bladder wall W as described herein.

As shown in FIGS. 2-6, the plication device 14 can be constructed in a generally rivet-shaped configuration to define a first head portion 20, an intermediate body member 22, and a second head portion 24. The head portions 20, 24 and/or the intermediate body member 22 can be constructed of a polymer metal material such the head portions 20, 24 are hingedly or pivotally displaceable or movable relative to the body member 22. Such a hingable configuration facilitates substantial collapse of the plication device 14 onto itself to facilitate deployment and placement. In certain embodiments, this movement or adjustment is made possible by the material construction itself. For instance, the body member 22, or a region or juncture 25 at which the head portions 20, 24 connect to the body member 22, can be generally flexible or constructed of a thin material (polymer or metal), thereby providing a level of “give” or hinging action at the juncture, or a portion of the body member 22.

In other embodiments, the body member 22 can include a living hinge portion 22a at or proximate the junctures 25 where the body member 22 meets the head portions 20, 24. Further, various embodiments of the plication device 14 can include movable ball, or ball and socket, mechanisms or features 22b to facilitate hinging of the head portions 20, 24 relative to the body member 22.

Various other embodiments can include an attachment feature 23 adapted to selectively receive an elongate member 27, such as a wire, rod or like member, to facilitate control over the plication device 14 during deployment and positioning. The member 27 can be generally rigid, partially flexible, partially rigid, or take on like constructs and characteristics to facilitate use as the means of controlling and moving the plication device 14.

The plication device 14 and its components can take on a variety of shapes and sizes, and can be constructed of any compatible metal, polymer or like material having generally flexible or rigid characteristics. Various sized plication devices 14 can be used to provide different spacing options between the head portions 20, 24, thereby allowing for larger or smaller tissue or bladder wall folds therebetween.

In certain embodiments, as shown in FIG. 6, the plication device 14 can include one or more adjustment features 30, enabling a user (e.g., physician) to adjust the spacing between the respective head portions 20, 24 to correspondingly adjust the compression on the tissue trapped therebetween during the plication folding process, and to allow for various sized tissue or bladder wall folds during the procedure. This may be important to ensure that the plicated tissue is secure, and to ensure that there is no leaking from the bladder through any tissue puncture created during the procedure. In one embodiment, the adjustment feature 30 can include a plurality of teeth or angled portions 32 provided along all or a portion of the body member 22. These teeth portions 32 are adapted to engage with corresponding or matingly compatible portions or like features in an aperture 34 of one or both of the head portions 20, 24 (e.g., head portion 24). In general, this mating teeth configuration will enable sliding or otherwise moving one of the head portions (e.g., head portion 24) closer to the other head portion (e.g., head portion 20) along the body member 22 to reduce the space therebetween (and with tissue therebetween as well), but the moving head portion (e.g., head portion 24) will not move back in the opposite direction to increase the distance between the heads or loosen the compression on the trapped fold of tissue.

Various embodiments can include other adjustment features 30 to permit adjustment of the distance between the head portions 20, 24, before or during the procedure. For instance, the body member 22 can include a threaded portion adapted to threadably engage with one or both of the head portions 20, 24 such that a screw or like interface (or one of the head portions 20, 24 itself) can be turned to selectively move the respective head portion 20, 24 (e.g., head portion 24) closer to or farther from the opposing head portion (e.g., head portion 20). Other embodiments can include one or more biasing members, such as a spring, along a portion of the body member 22 to provide selective biasing adjustment of the distance between the head portions 20, 24. Various fasteners, linear adjustment devices and mechanisms, and like mechanisms, devices and techniques known to those of skill in the art can be employed to provide the disclosed distance adjustment between the head portions 20, 24.

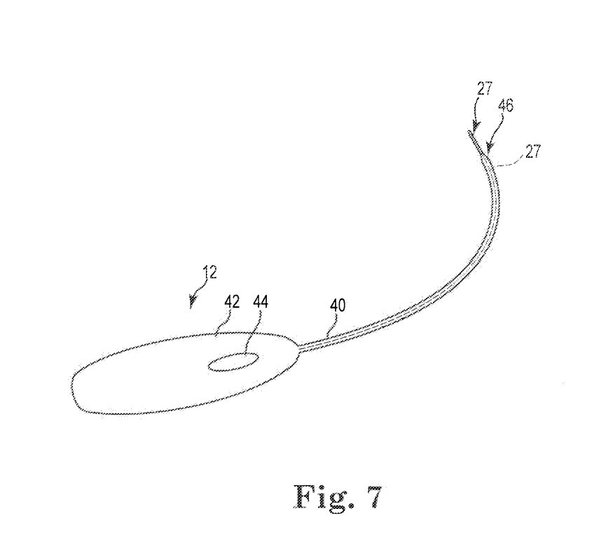

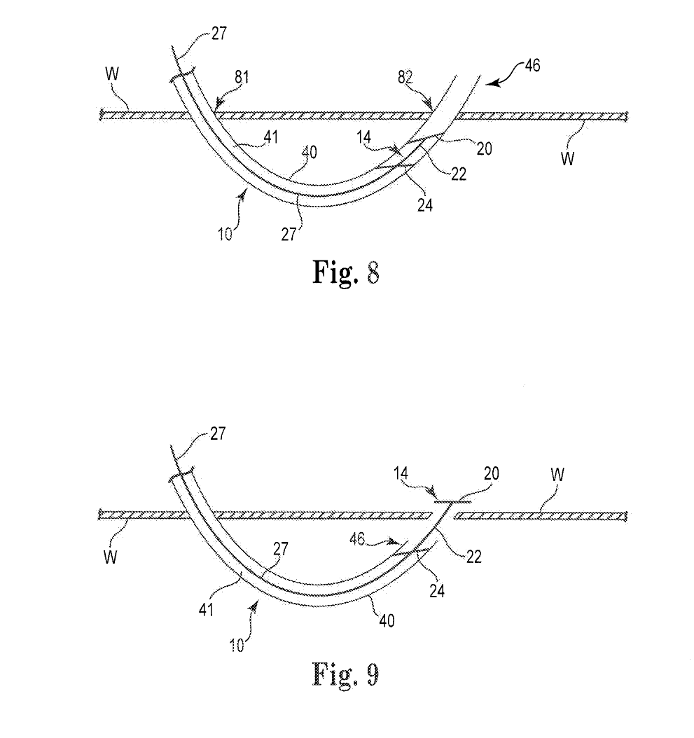

As shown in FIGS. 7-11, embodiments of a procedure and devices of the system 10 can be utilized to treat defects in the described internal sphincteric mechanism to resolve SUI in the patient. For instance, as shown in FIG. 8, the introducer needle device 12 can include a needle portion 40 having an internal lumen 41 adapted to receive and facilitate deployment of the plication device 14. The needle device 12 can include a handle portion 42 having an actuator 44. The actuator 44 can be in operable communication with the elongated member 27, controlling extension and retraction of the member 27. The needle device 12 can include blunt or piercing tips, and other features and mechanisms to facilitate device deployment and use.

In use, the plication device 14 is received in the lumen 41 of the needle portion 40 of the device 12 (e.g., generally collapsed) for use during the plication procedure. The member 27 is attached to one of the head portions 20, 24, or another portion of the plication device 14, to facilitate movement of and control of the device 14. The distal end or tip 46 of the needle device 12 can be advanced or pierced through a first bladder wall portion B1, traverse through a length of the bladder (within the interior of the bladder), and out through a second bladder wall portion B2, as shown in FIG. 8.

As shown in FIG. 9, the plication device 14 can then be deployed out the distal end 46 of the needle device 12. The first head portion 20 can be extended out (pushed by member 27) from the second bladder wall portion B2 to rest or engage against the outside surface of the bladder wall W. Various protrusions, anchors, or material constructs can be employed to facilitate engagement with the bladder wall.

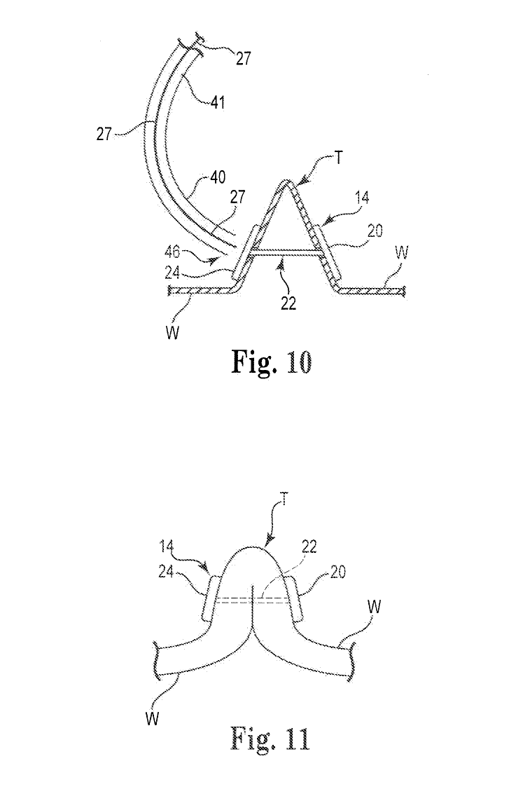

Upon deploying the first head portion 20, the needle device 12 can be backed out such that the second head portion 24 remains secured within the needle lumen 41 until the distal end 46 exists out through the first bladder wall portion B1, as shown in FIGS. 9-10. At this point, the second head portion 24 can be deployed from the distal end 16 for securement or engagement against the outside surface of the bladder proximate the first bladder wall portion B1. The member 27 can be removed from the second head portion 24, e.g., via a pulling force, cutting, snap disengagement, unlatching, twisting, and the like. As such, the bladder wall, between bladder wall portions B1 and B2, can be cinched or folded onto itself to define the plicated or folded tissue T to reduce slack and tighten up the target bladder wall region. This procedure can be repeated for multiple target sites or bladder wall portions, including those at or proximate the internal sphincter mechanism, using additional plication devices 14.

This system 10 and surgical procedure has many advantages, including not requiring an incision (surgical entry through the urethra), reduced invasiveness, direct treatment of ISD, and less anesthesia and recovery time requirements for the patient than other surgical procedures.

While embodiments of the system 10 are described in relation to treating ISD and SUI, other applications for treating other pelvic or applicable tissue areas are envisioned as well.

The various systems, devices, tools, features and methods disclosed hereinbelow and directed to pelvic implant and repair systems (e.g., for male and female), are envisioned for use with the present invention, including those disclosed in U.S. Pat. Nos. 7,500,945, 7,407,480, 7,351,197, 7,347,812, 7,303,525, 7,025,063, 6,691,711, 6,648,921, and 6,612,977, International Patent Publication Nos. WO 2008/057261 and WO 2007/097994, and U.S. Patent Publication Nos. 2010/0105979, 2002/151762 and 2002/147382. Accordingly, the above-identified disclosures are fully incorporated herein by reference in their entirety.

The systems 10, their various components, structures, features, materials and methods may have a number of suitable configurations as shown and described in the previously-incorporated references. Various methods and tools for introducing, deploying, anchoring and manipulating devices to treat incontinence and prolapse as disclosed in the previously-incorporated references are envisioned for use with the present invention as well. Further, the system and its components or structures can be constructed of known and compatible materials know to those skilled in the art, including metals, polymers, and the like.

All patents, patent applications, and publications cited herein are hereby incorporated by reference in their entirety as if individually incorporated, and include those references incorporated within the identified patents, patent applications and publications.

Obviously, numerous modifications and variations of the present invention are possible in light of the teachings herein. It is therefore to be understood that within the scope of the appended claims, the invention may be practiced other than as specifically described herein.

What is claimed is:

1. A plication system for treating incontinence, comprising:
a needle delivery device including a generally hollow needle having a distal end portion, wherein the distal end portion of the generally hollow needle is adapted to traverse the urethra, into the bladder, and through a target tissue of the bladder wall; and
at least one plication device having a first head portion, a body portion, and a second head portion, wherein the at least one plication device is deployable from the distal end portion of the generally hollow needle to plicate the target tissue of the bladder wall.

2. The system of claim 1, wherein the target tissue is proximate the inner urethral sphincter.

3. The system of claim 1, wherein the at least one plication device includes two or more plication devices.

4. The system of claim 1, further including an extending member selectively attached to the second head portion of the at least one plication device and adapted to direct movement of the at least one plication device relative to the generally hollow needle.

5. The system of claim 4, wherein the extending member is a wire.

6. The system of claim 1, wherein the first head portion and the second head portion of the at least one plication device are pivotally connected to the body portion.

7. The system of claim 1, wherein the first head portion and the second head portion of the at least one plication device are hingedly connected to the body portion.

8. A bladder wall plication system for treating incontinence, comprising:
a needle delivery device including a generally hollow needle having a distal end portion, wherein the distal end portion of the generally hollow needle is adapted to traverse the urethra and into the bladder to pierce through a target region of the bladder wall; and
at least one plication device having a first head portion, a second head portion, an adjustment feature, and a body member generally providing a spacing between the first and second head portions, wherein the at least one plication device is deployable from the distal end portion of the generally hollow needle to plicate the target region of the bladder wall such that the adjustment feature facilitates adjustment of the spacing between the first and second head portions.

9. The system of claim 8, wherein the target region of the bladder is proximate the inner urethral sphincter.

10. The system of claim 8, wherein the at least one plication device includes two or more plication devices.

11. The system of claim 8, further including an extending member selectively attached to the second head portion of the at least one plication device and adapted to direct movement of the at least one plication device relative to the generally hollow needle.

12. The system of claim 11, wherein the extending member is a wire.

13. The system of claim 8, wherein the first head portion and the second head portion of the at least one plication device are pivotally connected to the body member.

14. The system of claim 8, wherein the first head portion and the second head portion of the at least one plication device are hingedly connected to the body member.

15. The system of claim 8, wherein the adjustment feature includes a plurality of teeth provided along at least a portion of the body member such that the second head portion is slidable along the body member for selective securement a distance closer to the first head portion.

16. The system of claim 15, wherein the plurality of teeth are angled teeth.

17. The system of claim 8, further including an extending wire member selectively attached to an attachment feature of the second head portion of the at least one plication device to facilitate movement of the at least one plication device.
